(19) United States
(12) Patent Application Publication
(10) Pub. No.: US 2009/0048486 A1
(43) Pub. Date: Feb. 19, 2009

(54) Distal Tip for an Endoscope

(75) Inventor: Vihar C. Surti, Winston-Salem, NC (US)

Correspondence Address: BRINKS HOFER GILSON & LIONE/CHICAGO/COOK, PO BOX 10395, CHICAGO, IL, 60610, US

(73) Assignee: Wilson-Cook Medical Inc., Winston-Salem, NC (US)

(21) Appl. No.: 12/186,800

(22) Filed: Aug. 6, 2008

Related U.S. Application Data
(60) Provisional application No. 60/954,618, filed on Aug. 8, 2007.

(51) Int. Cl. A61B 1/012

(52) U.S. Cl. 600/127

(57) ABSTRACT

A system configured for endoscopic treatment of tissue is provided. The system includes an elongate shaft having a proximal portion, a distal portion and a working channel extending at least partially through the shaft. The elongate shaft further includes a longitudinally extending central axis. A distal end of the distal portion of the elongate shaft includes a distal tip having a curvilinear portion. The curvilinear portion is configured to dilate a stricture in a gastrointestinal tract for passing a portion of the system through the stricture to the tissue. The distal tip includes a working channel port connected to the working channel.

### RELATED APPLICATIONS

This application claims the benefit of U.S. Provisional Application No. 60/954,618, filed Aug. 8, 2007, which is incorporated by reference herein in its entirety.

### TECHNICAL FIELD

This invention generally relates to medical devices, and more particularly to endoscopes having an improved distal tip.

### BACKGROUND

Endoscopic devices and endoscopic surgeries have been commonly used for various procedures, typically in the abdominal area. The advantages of minimally invasive surgery performed with the help of an endoscope are well known and understood in the medical field. As a result, there have been a growing number of devices for use with endoscopes for delivering, for example, diagnostic, monitoring, treatment, operating instruments, tools, and accessories into the observation field and working space of the physician's endoscope. A wide range of applications have been developed for the general field of endoscopes including by way of example the following: arthroscope, angioscope, bronchoscope, choledochoscope, colonoscope, cytoscope, duodenoscope, enteroscope, esophagogastro-duodenoscope (gastroscope), laparoscope, laryngoscope, nasopharyngo-neproscope, sigmoidoscope, thoracoscope, and utererscope (individually and collectively, “endoscope”). In addition, surgical procedures for entry into the peritoneal cavity through natural orifices with out skin incisions may be performed using an endoscopic device to gain entry to the peritoneum. This type of procedure is referred to as Natural Orifice Translumenal Endoscopic Surgery (NOTES).

Typically, a conventional endoscope generally is an instrument with a light source and image sensor for visualizing the interior of an internal region of a body. In order to form an image of the scene under observation, a light source and image sensor are features that may be provided at or near the distal end portion of an insertion section of the endoscope that is to be inserted into the body. Endoscopes may also incorporate additional features for observation or operation within the body, such as a working channel for passing diagnostic, monitoring, treatment, or surgical tools through the endoscope, where the working channel has an opening located at the distal end portion of the endoscope to provide access to a site within the patient.

Currently available endoscopes have drawbacks, especially for navigating through a passageway of a patient. In particular, conventional endoscopes have a blunt distal end face where the light source and imaging sensor terminate. Blunt portions, such as the existing distal face, can cause a variety of difficulties during medical procedures. For example, when an endoscope is navigated through a tight passageway or stricture, the surrounding tissues can catch or snag on these blunt portions of the distal end of the endoscope. In addition, the blunt portion of the distal end of the endoscope can make it difficult for a physician to smoothly traverse a stricture. The progress of the endoscope through the passageway may further irritate the tissue through which the endoscope passes and catches. In addition, the endoscope includes an end that typically has a larger diameter than the diameter of the stricture through which the endoscope is navigated. For example, the end of the endoscope may be inserted through a small incision in a natural orifice having a smaller diameter than the diameter of the end of the endoscope during the NOTES procedure. Thus, there is a need for an endoscope that improves or resolves these drawbacks.

### SUMMARY OF THE INVENTION

Accordingly, it is an object of the present invention to provide an endoscope having an improved distal end portion for atraumatically navigating and dilating a passageway that resolves or improves on one or more of the above-described drawbacks.

The foregoing object is obtained by providing a system configured for endoscopic treatment of tissue. The system includes an elongate shaft having a proximal portion, a distal portion and a working channel extending at least partially through the shaft. The elongate shaft further includes a longitudinally extending central axis. A distal end of the distal portion of the elongate shaft includes a distal tip having a curvilinear portion. The curvilinear portion is configured to dilate a stricture in a gastrointestinal tract for passing a portion of the system through the stricture. The distal tip includes a working channel port connected to the working channel, an imaging port and a lighting port.

In another aspect, a cap configured for used with an endoscope is provided. The cap includes a proximal portion and a distal portion. The proximal portion is configured for operably connecting to a distal end portion of the endoscope. The distal portion of the cap includes a curvilinear portion having a working channel port, an imaging port, and a lighting port. The curvilinear portion is configured to dilate a stricture in a gastrointestinal tract for passing a portion of the endoscope through the stricture.

In another aspect of the present invention, a method of endoscopically treating a tissue is provided. The method includes providing an elongate shaft having a proximal portion, a distal portion and a distal end having a distal tip. The distal tip includes a curvilinear portion having a working channel port, an imaging port, and a lighting port. The method further includes advancing the distal end to a stricture in a patient, the stricture having a diameter that is narrower than a diameter of the distal end, engaging the stricture with the curvilinear portion and dilating the stricture to pass the distal end through the stricture to access the tissue.

The foregoing paragraphs have been provided by way of general introduction, and are not intended to limit the scope of the following claims. The presently preferred embodiments, together with further advantages will be best understood by reference to the following detailed description taken in conjunction with the accompanying drawings.

### BRIEF DESCRIPTION OF THE DRAWINGS

FIG. 1 is a side view of an endoscope of the present invention having a curvilinear distal end;

FIG. 2 is a partial side view of through the distal portion of the endoscope shown in FIG. 1;

FIG. 3 is a cross-sectional view of a distal portion of the endoscope shaft shown in FIG. 1 along line A-A;

FIG. 4 is a side sectional view of the distal portion of the endoscope shown in FIG. 1 having a curvilinear end;

FIG. 5 is a side sectional view of an alternative distal portion of the endoscope shown in FIG. 1 having a curvilinear end;

FIG. 6 is a perspective view of an alternative distal portion of an endoscope having a removable cap at the distal end;

FIG. 7 is a perspective view of a distal portion of an endoscope having a removable distal end having an asymmetric end; and

FIG. 8 is a perspective view of an alternative distal portion of an endoscope having an asymmetric end.

### DETAILED DESCRIPTION OF THE EMBODIMENTS

The present invention relates to medical devices and, in particular, to endoscopic access devices. The invention is described with reference to the drawings in which like elements are referred to by like numerals. The relationship and functioning of the various elements of this invention are better understood by the following detailed description. However, the embodiments of this invention are not limited to the embodiments illustrated in the drawings. It should be understood that the drawings are not to scale, and in certain instances details have been omitted which are not necessary for an understanding of the present invention, such as conventional fabrication and assembly. As used herein the terms comprise(s), include(s), having, has, with, contain(s) and variants thereof are intended to be open ended transitional phrases, terms, or words that do not preclude the possibility of additional steps or structure.

As used in the specification, the terms proximal and distal should be understood as being in the terms of a physician using the endoscope. Hence the term distal means the portion of the endoscope which is farthest from the physician and the term proximal means the portion of the endoscope which is nearest to the physician.

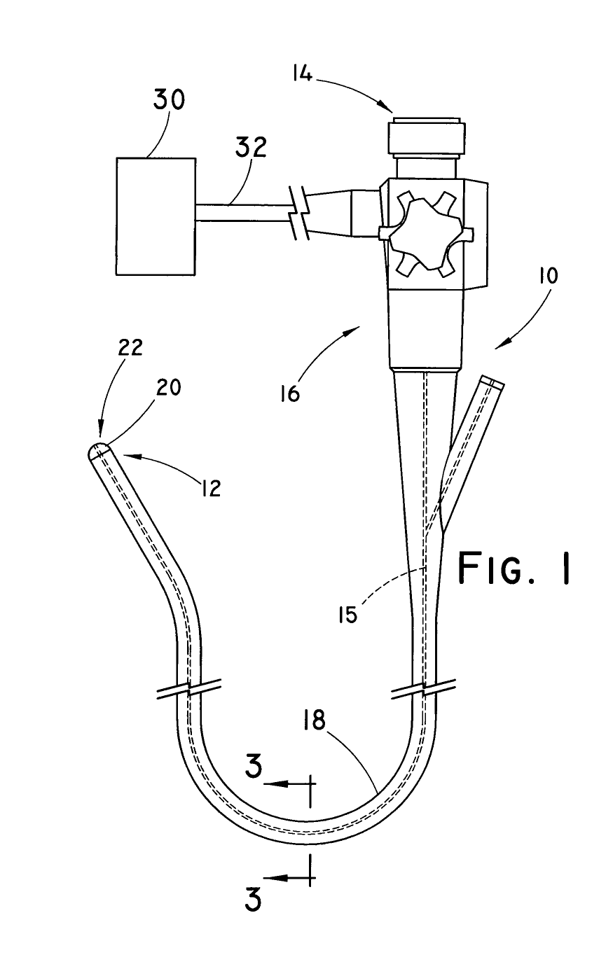

FIG. 1 illustrates an endoscope 10 according to the present invention. The endoscope 10 may be a rigid endoscope or a flexible endoscope, as is generally known in the art. The endoscope 10 may be designed for single use or the endoscope may be reusable. The endoscope 10 includes a distal portion 12 and a proximal portion 14. The endoscope 10 further includes a handle portion 16 at the proximal portion 14 and an elongate shaft 18 extending distally from the handle portion 16 to a distal end 20 of the endoscope 10. At least one lumen 15 extends at least partially through the shaft 18. The lumen 15 may be a working channel of the endoscope for passage of accessories through the channel to the target site. The endoscope 10 includes a viewing system 22 having an imaging system and a lighting system (described in more detail below).

The endoscope 10 is operably connected to a control unit 30 via conduit 32. The control unit 30 provides image processing for image display on a monitor (not shown) and includes a data storage unit for storing programming software as well as digital representation of the images. The control unit 30 also provides a power source, fluids, vacuum source and the like, to the endoscope 10. Any type of control unit known to one skilled in the art may be used with the endoscope of the present invention.

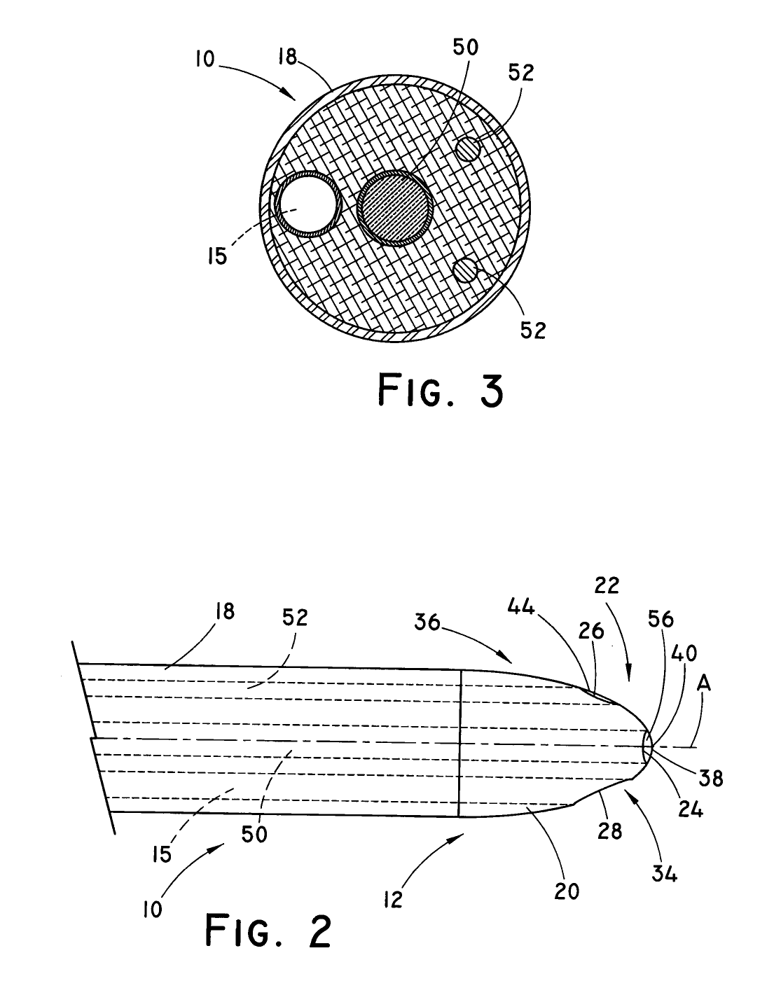

An enlarged side view of the distal portion 12 of the shaft 18 is shown in FIG. 2. The distal portion 12 includes the distal end 20 having a curvilinear profile 34. The term “curvilinear”, as used herein, refers to a generally curved profile, which may include hemispherical, elliptical or arch-shaped profiles and the like. This configuration provides for smooth entry into body passageways having strictures or blockages. The curvilinear profile 34 may be used to contact and dilate the stricture to facilitate passage of the distal portion 12 of the endoscope 10 through the stricture. The curvilinear profile 34 may contact and dilate strictures such as sphincters, incisions through a natural orifice, or abnormal outgrowths in a passageway, such as a tumor. The curvilinear profile 34 also prevents or decreases the incidence of the distal end 20 inadvertently snagging and thereby unnecessarily traumatizing surrounding body tissues. The distal end 20 may also include a conical portion 36 that tapers inward from the shaft 18 to a distal tip 38 as shown in FIG. 2. The conical portion 36 may also be used to facilitate the dilation of the stricture for passage of the distal portion 12 through the stricture.

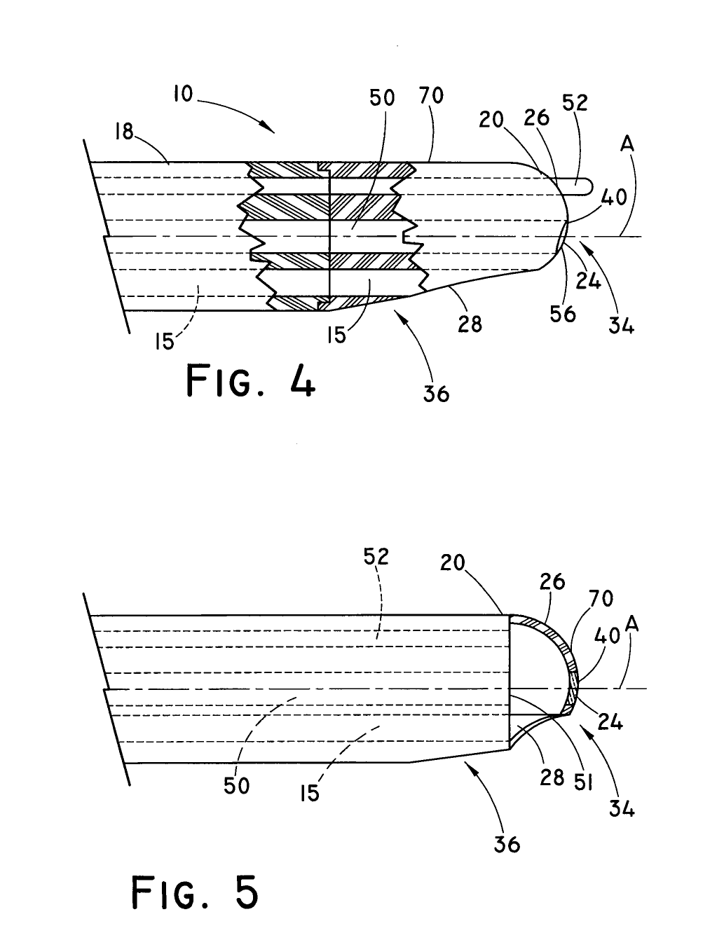

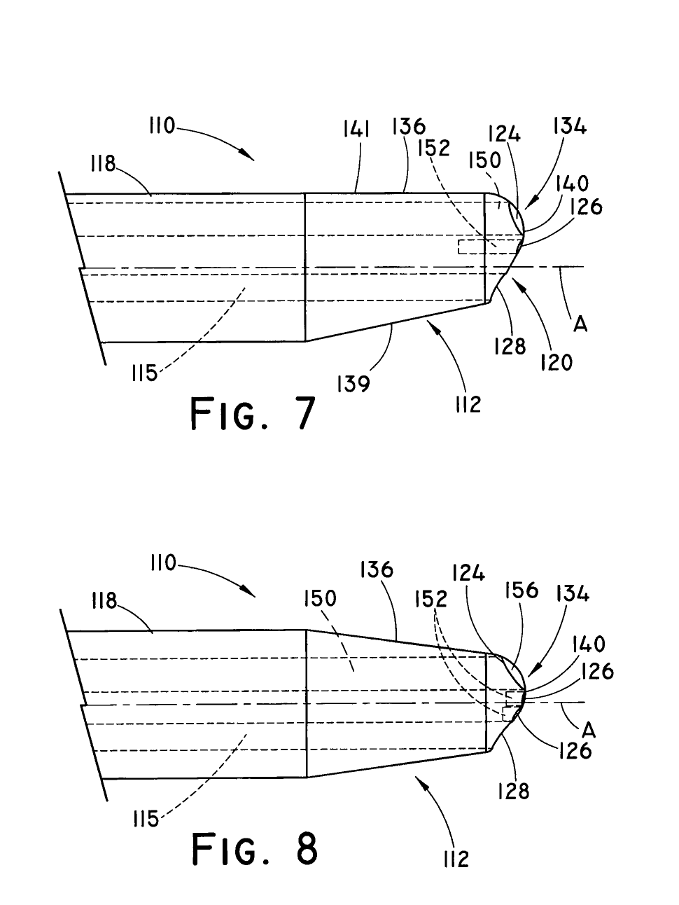

In some embodiments the distal end 20 may be a hemispherically-shaped dome as shown in FIGS. 4 and 5. In some embodiments, the distal end 20 may be asymmetric as shown in FIGS. 7 and 8. For example, the distal end 20 may be asymmetric with respect to features, including, but not limited to, the curvilinear portion 34, the conical portion 36, an apex 40 at the distal tip 38 or any of the openings or ports extending to the distal end 20, or the central axis. Combinations of these features may also be provided to form an asymmetrical distal end 20.

As shown in FIG. 2, the distal end 20 may include an imaging port 24, a lighting port 26 and a working channel port 28. Additional ports/openings may also be included. The placement of the ports 24, 26 and 28 may be provided in any configuration. For example, in some embodiments, the imaging port 24 may be centrally positioned for optimal image viewing as discussed below. Alternatively, the imaging port 24 may be off-set for optimal viewing. The ports 24, 26 and 28 may be provided with a periphery having a variety of configurations. For example, one or more of the ports may be provided with a generally circular or oval periphery at the surface of the curvilinear portion 34. The shape of the periphery of the ports may be altered by changing the shape of a lumen connected to the port and by the position of the port on the curvilinear portion. For example, a centrally located port may have a circular periphery and a non-centrally positioned port may have a more oval periphery due to the position of the port on the curvilinear portion and/or the conical portion. The shape of the periphery of the port, for example, the working channel port 28, may be optimized to provide a smooth surface on the curvilinear portion 34 around the port 28. In some embodiments, the ports 26 and 28 may be provided with a lens or a filter covering the ports 24, 26 as discussed below.

As shown in FIG. 2, the endoscope 10 may also include an imaging system 50, a lighting system 52 and the lumen 15 that may be provided as a working channel for use with of any kind of diagnostic, monitoring, treatment, or surgical tools that may be provided to a patient through the endoscope. In some embodiments, a flushing port and associated lumen (not shown) may also be provided for cleaning the imaging port 24 and the lighting port 26. A cross-sectional view through the shaft 18 of the endoscope 10 taken along line A-A of FIG. 1 is shown in FIG. 3. The shaft 18 may include the lumen 15, the imaging system 50 and the lighting system 52 extending therethrough. Alternatively, the imaging system 50 or the lighting system 52 may be provided on the distal end 20 without extending through the shaft 18. See for example, the lighting system 52 shown in the distal end 20 of the shaft 18 in FIGS. 7 and 8. As shown in FIGS. 2-4, the imaging port 24 may be centrally positioned with respect to a central longitudinal axis A through the endoscope 10. The lighting port 26 and working channel port 28 may be arranged around the imaging port 24 in any configuration.

The imaging system 50 may be disposed proximal to the imaging port 24. The imaging system 50 may be any kind of imaging system suitable for viewing a body passageway. The imaging system 50 may include one or more optical lenses and a solid-state imaging sensing device such as a charged coupled device (CCD) or a complementary metal-oxide-semiconductor (CMOS) image sensor that produces electronic signals representative of an image of the tissue in front of the imaging port 24. The imaging system 50 may include optical fibers that are optically coupled to an image sensor. As shown in FIG. 2, a lens 56 may cover the imaging port 24. The lens 56 may be formed as part of the curvilinear portion 34 or the lens 56 may be a separate lens that is fitted into the imaging port 24. The lens 56 may be sized and shaped to provide the optimal image through the curvilinear distal end 20. The lens 56 or a portion thereof may be planar to improve the optical image while still having an overall curvilinear distal end 20.

The lighting system 52 may be disposed proximal to the lighting port 26 as shown in FIG. 2. The lighting system 52 may be any kind of lighting system known to one skilled in the art. For example, the lighting system may include one or more lenses and one or more lighting sources. The lighting source may include emitting diodes (LEDs) or liquid crystal displays (LCDs). The LEDs may be high intensity white light sources or they may be colored light sources such as red, green and blue or ultra-violet LEDs. In some embodiments, the lighting system 52 may include a movable lighting bundle that may be extended distally through the lighting port 26 to provide illumination closer to the tissue. In some embodiments, the lighting system 52 may include one or more LEDs mounted at the distal end 20 and electrically connected to the control unit 30. Cooling mechanisms for the lighting source may also be provided (not shown).

In the embodiment shown in FIG. 2, the lighting port 26 may include a lighting lens or filter 44 at the tip of the distal end 20 of the shaft 18. The lighting lens 44 may be formed as part of the curvilinear portion 34 or the lens 44 may be a separate lens that is fitted into the lighting port 26. A plurality of lighting ports 26 with lighting systems 52 may be provided at the distal end 20. The lighting ports 26 may be positioned with respect to the imaging port 24 to provide optimal illumination for the imaging system 50.

As shown in FIGS. 4 and 5, the curvilinear portion 34 may be provided as a hemispherically-shaped dome. As shown in FIG. 4, the imaging system 50 may extend to the distal tip 40 of the endoscope 10 wherein the lens 56 is positioned in the imaging port 24. The imaging system 50 is shown positioned along the central longitudinal axis A. The lighting system 52 may be provided as a movable lighting bundle wherein the lighting system 52 may extend through the lighting port 26 and extend distally out from the distal end 20 of the endoscope 10 to illuminate the tissue. The distal end portion 20 may be provided as a removable cap portion 70 that may be connected together with the shaft 18. The connection of the cap 70 to the shaft 18 may be a snap fit connection, threaded connection and the like. The cap 70 may fit over the end of the shaft 18 so that the imaging system 50 is provided with the shaft 18 and the cap 70 slides on to the shaft 18 until the imaging system 50 is positioned at the lens 56. Alternatively, the cap 70 may include a plurality of lumens that terminate in the ports 24, 26 and 28 and that connect to respective lumens provided in the shaft 18. In this configuration, the imaging system 50 may be positioned after the cap 70 is position on the shaft 18. The lighting system 52 may also be extended to the lighting port 26 after the cap 70 is positioned. The lighting system 52 may be extended further distally when the distal end 20 of the endoscope 10 is positioned at the tissue location. The lighting port 26 and the working channel port 28 are off-set from the central axis A.

In some embodiments, for example as shown in FIG. 5, the imaging system 50 may be recessed from the distal tip 40 and may have a planar surface 51 at the end of the imaging system 50 that is proximal to the distal end 20 of the endoscope 10. The lighting system 52 may also be recessed from the distal end 20. The embodiment shown in FIG. 5 may include the cap 70 that provides the curvilinear portion 34. The cap 70 includes the working channel port 28 that aligns with the lumen 15 in the shaft 18. The cap 70 may be provided as a transparent material such as a polycarbonate, an acrylic material or any material that provides proper imaging capabilities. The cap 70 may be optimized for transmission of the imaging system 50 and the lighting system 52. The imaging and lighting ports 24, 26 may be integrally formed in the cap 70 and may additionally include the lenses 44 and/or 56. For example, the lens 44 may be a wide angle lens that is configured to operably connect with the imaging system 50.

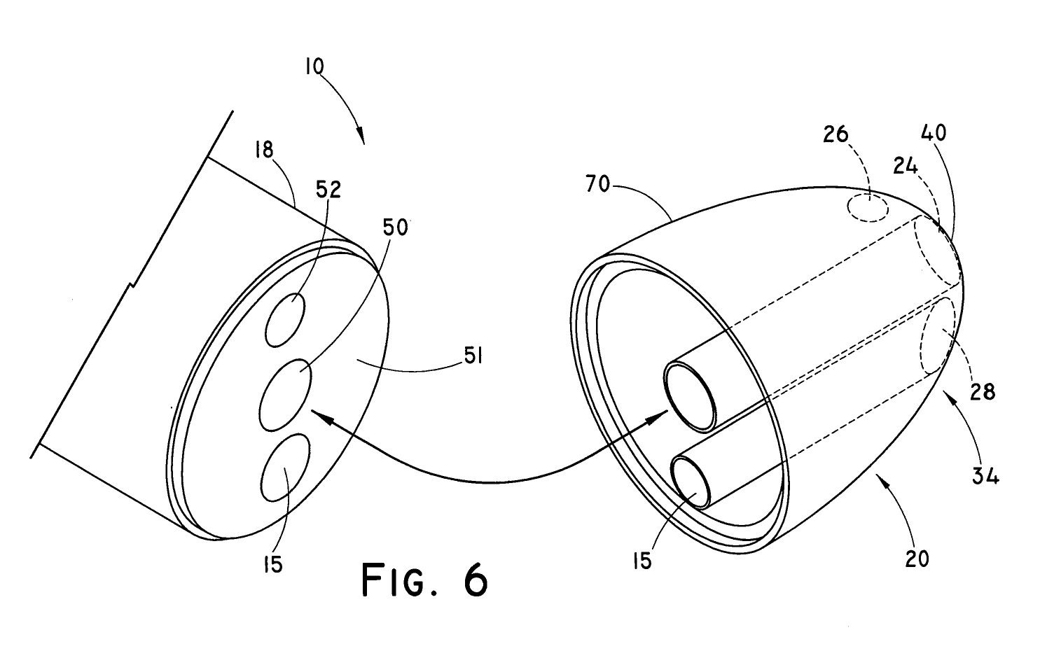

FIG. 6 illustrates the cap 70 removed from the shaft 18 of the embodiment shown in FIG. 5. As shown, the shaft 18 includes the planar surface 51 where the imaging system 50 and the lighting system 52 may terminate. The lumen 15 is also shown in the shaft 18 of the endoscope 10. The cap 70 includes the curvilinear portion 34 at the distal end 20. The imaging port 24 is shown at the apex 40 of the curvilinear portion 34. As discussed above, a lens or a series of lenses may be included between the imaging port 24 and the imaging system 50 at the planar surface 51. The cap 70 also includes the lighting port 26 and the working channel port 28. The lumen 15 operably connects with the working channel port 28.

In some embodiments, the hemispherically-shaped dome at the curvilinear portion 34 may be formed integrally with the shaft 18 to form the distal end 20. The imaging port 24, the lighting port 26 and the working channel port 28 may also be provided with the integrally formed distal end 20 of the endoscope 10.

An alternative embodiment of a distal end 120 of a shaft 118 of an endoscope 110 is shown in FIGS. 7 and 8. The endoscope 110 differs from the endoscope 10 described above in that the endoscope 110 includes an asymmetric curvilinear portion 134 at the distal end 120 of the shaft 118. As shown, an apex 140 is off-set from the central longitudinal axis A of the shaft 118. Similar to the embodiments described above, the distal end 120 may include an imaging port 124, a lighting port 126 and a working channel port 128. The endoscope 110 may also include an imaging system 150, a lighting system 152 and a lumen 115 that may be provided as a working channel for use with of any kind of diagnostic, monitoring, treatment, or surgical tools that may be provided to a patient through the endoscope. A flushing port and associated lumen (not shown) may also be provided for cleaning the imaging port 124 and the lighting port 126. In the embodiment shown in FIG. 7, the imaging port 124 is off-set from the central longitudinal axis A. The imaging port 124 may be provided at the apex 140 with the imaging system 150 provided proximal to the imaging port 150. The imaging port 124 may be positioned for optimal imaging as described above.

The lighting port 126 is shown positioned adjacent the imaging port 124. The lighting system 152 may be one or more LEDs that are mounted in the distal end 120 and that do not extend through the shaft 118. The lighting system 152 may include a portion that is electrically connected to the control system 30. Alternatively, the lighting system 152 may be battery operated. The working channel port 128 may also be off-set from the central axis A. The working channel port 128 is connected to the lumen 115 and provides access to the patient for any tool that may be used with the endoscope 110. The distal end 120 may include an asymmetric conical portion 136 wherein a portion 139 of the conical portion 136 is angled inward from the shaft 118 to the distal end 120 and a portion 141 of the conical portion extends parallel to the shaft 118 to the distal end 120. The conical portion 136 and/or the distal end 120 may be provided as a cap or the conical portion 136 and the distal end 120 may be integrally formed with the shaft 118.

An asymmetric curvilinear portion 134 of the endoscope 110 is also shown in FIG. 8. FIG. 8 illustrates the endoscope 110 having the apex 140 off-set from the central longitudinal axis A and the conical portion 136 being angled inward from the shaft 118 to the distal end 120. Similar to the embodiment shown in FIG. 7, this embodiment includes the imaging port 124 that is off-set from the central axis A. The imaging port 124 may include a planar portion such as a lens 156 to optimize the optics through the imaging system 150 that is disposed proximal to the imaging port 124. Two lighting ports 126 and two lighting systems 152 are shown at the distal end 120 positioned adjacent to the imaging port 124. The working channel 128 is provided on the distal end 120 and connects to the lumen 115.

The above Figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

1. A system configured for endoscopic treatment of tissue comprising:
an elongate shaft comprising a proximal portion, a distal portion and a working channel extending at least partially through the shaft, and a longitudinally extending central axis; and
a distal end of the distal portion comprising a distal tip, the distal tip comprising:
a curvilinear portion having a working channel port connected to the working channel, the curvilinear portion configured to dilate a stricture in a gastrointestinal tract for passing a portion of the system through the stricture.

2. The system of claim 1, wherein the distal tip and the distal portion are a unitary construction.

3. The system of claim 1, wherein the curvilinear portion comprises a removable cap operably connected to the distal portion.

4. The system of claim 1, wherein the curvilinear portion is asymmetric.

5. The system of claim 1, wherein the distal end further comprises a conical region proximal to the distal tip.

6. The system of claim 5, wherein the conical region is asymmetric.

7. The system of claim 1, wherein the curvilinear portion further includes an imaging port, a lighting port or both.

8. The system of claim 7, wherein the imaging port is off-set from the central axis.

9. The system of claim 7, wherein the imaging port is provided at an apex of the distal tip.

10. The system of claim 7, further comprising an imaging system disposed proximal and operably connected to the imaging port.

11. The system of claim 7, wherein the imaging system further comprising a lens disposed at the imaging port.

12. The system of claim 7, further comprising a lighting system disposed proximal and operably connected to the lighting port.

13. The system of claim 12, wherein the lighting system comprises a fiber optic bundle, the fiber optic bundle being adapted for advancing through the lighting port.

14. The system of claim 1, wherein the working channel port comprises a non-circular periphery.

15. The system of claim, 1 wherein the curvilinear portion includes a planar surface.

16. A cap configured for use with an endoscope, the cap comprising:
a proximal portion and
a distal portion, the distal portion comprising a curvilinear portion having a working channel port, the curvilinear portion configured to dilate a stricture in a gastrointestinal tract for passing a portion of the system through the stricture;
wherein the proximal portion is adapted to contact a distal end portion of an endoscope.

17. The cap of claim 16, wherein the curvilinear portion is asymmetric.

18. The cap of claim 16, wherein the cap further comprises a lens at an imaging port configured for transmitting an image to an image system of the endoscope.

19. A method for endoscopically treating tissue, the method comprising:
providing an elongate shaft having a proximal portion, a distal portion, and a distal end comprising a distal tip, the distal tip comprising a curvilinear portion having a working channel port;
advancing the distal end to a stricture in a patient, the stricture having a diameter that is narrower than a diameter of the distal end;
engaging the stricture with the curvilinear portion; and
dilating the stricture to pass the distal end through the stricture to access the tissue.

20. The method of claim 19, wherein the curvilinear portion is asymmetric.

21. The method of claim 19, wherein the curvilinear portion comprises a removable cap operably connected to the distal portion of the shaft.

22. The method of claim 19 wherein the stricture comprises an incision through a natural orifice and the end is advanced through the incision to access an abdominal cavity.
